(19) United States
(12) Patent Application Publication
(10) Pub. No.: US 2020/0353512 A1
(43) Pub. Date: Nov. 12, 2020

(54) RESTROOM SANITATION AND MONITORING SYSTEM

(71) Applicant: ALEXEI FOMINE, HOUSTON, TX (US)

(72) Inventor: ALEXEI FOMINE, HOUSTON, TX (US)

(21) Appl. No.: 16/920,473

(22) Filed: Jul. 3, 2020

Related U.S. Application Data
(63) Continuation-in-part of application No. 16/294,784, filed on Mar. 6, 2019.

(51) Int. Cl. B08B 7/00; A47K 10/44; B25J 11/00; B08B 13/00; A61L 2/24; A61L 2/10; A61L 9/12

(52) U.S. Cl. CPC B08B 7/0057; A47K 10/44; B25J 11/0085; A47K 2010/3266; A61L 2/24; A61L 2/10; A61L 9/122; B08B 13/00

(57) ABSTRACT

An electronic cleaning system includes a handle assembly configured to grasp a moistened tissue. A cartridge is included within a dispensing unit to store the tissue. A motor selectively releases the tissue from the cartridge. A control unit is configured to monitor and selectively control a plurality of devices that permit the hands-free cleaning and sanitizing of a defined area. The control unit processes and transmits data regarding the status of the defined area. A remote panel and auxiliary systems assist in cleaning and conveying information to users. The system permits for manually cleaning of surfaces in the defined area without the need to physically contact any of the cleaning tissue, chemicals, or surfaces therein. Additionally, the system includes UV light emitting systems to clean remote surfaces and the air. The user is separated from the potentially harmful surfaces and chemicals.

### CROSS REFERENCE TO RELATED APPLICATIONS

This application claims the benefit of and is a continuation in part of U.S. patent Ser. No. 16/294,784, filed 6 Mar. 2019, the contents of which is incorporated by reference herein in its entirety.

### BACKGROUND OF THE INVENTION

### 1. Field of the Invention

The present application relates generally to a system for cleaning surfaces and the air of a room, and more particularly to a sanitation and monitoring system for a restroom that is able to clean the air and surfaces, as well as include an assembly for dispensing moistened towelettes to a handle device for grasping and using the towelettes to directly contact and clean surfaces.

### 2. Description of Related Art

A concern for a large number of individuals is cleanliness of surfaces in homes and businesses that are frequented. Direct hand contact with unsanitary or unclean surfaces as well as cleaning chemicals used to clean the surfaces is undesirable. In one instance, a common area of concern is the bathroom, and in particular the toilet seat. Many methods and devices have been developed to ensure the proper cleanliness of the toilet. For example, a cleaning compound and fluids may be sprayed onto a surface from a spray bottle to clean or sanitize the surface. Upon application to the surface, a user often wipes the dirty surface with a rag, sponge, tissue, or other similar cleaning device. In this instance, the cleaning device requires user's hand to be in close proximity to the surface, often in direct contact with the cleaning surface and/or the cleaning tissue. The tissue then becomes quickly saturated, typically contacting with the user's hand, after which the device or tissue must be discarded, cleaned, or laundered. Although, some devices do protect the user from holding the cleaning tissue directly by hand during the cleaning, after the cleaning it still requires the user to grab the tissue by hand to discard it. After such use, a user may be required to wash his/her hands, which is inconvenient and inefficient.

It is understood that this method of cleaning a surface has multiple limitations and disadvantages. For instance, to perform the cleaning, the user's hand is in direct contact with the cleaning tissue prior to cleaning, during, or after it once the cleaning tissue needs to be discarded or cleaned. Hands are susceptible to contacting the dirty surface and the saturated areas of the cleaning tissue itself causing many users psychological distress and possible irritation.

As a result, some users aim to avoid such close contact with cleaning devices or surfaces, especially toilet seats, while many others completely avoid use of any publicly accessible toilets due to unsanitary conditions, as well as unavailability of effective cleaning devices. Additionally, many individuals have allergies or other harmful reactions and irritations from various cleaning products that may make contact with the cleaning compound hazardous. Others don't want to touch the cleaning tissue because of the odor emitted by the cleaning compound (i.e. alcohol smell). Furthermore, it can be difficult to estimate the amount of cleaning compound and cleaning device necessary to avoid contact with the cleaning compound yet assure a thorough cleaning.

These disadvantages are only amplified by the thought of unsanitary surfaces in commercial establishments: public toilets, handles, tables, hospitals and so forth. For example, in hospitals there may exist any number of viruses and bacteria, or even biological substances that need to be cleaned up from a surface (i.e. blood). It is undesirable for a user to come into contact with any of these. Cleaning becomes even more complicated since many cleaning devices are stored apart from the cleaning tissue, forcing user to travel to the tissue storage container each time a tissue refill is needed, increasing chances of contamination and contact with tissues, dirt, and cleaning chemicals.

Studies have shown that 70% of restroom users have unpleasant restroom experiences, while 23% of those customers leave an establishment without completing business. Restroom spaces are currently provided and built to meet only basic human needs. They include a toilet, toilet paper, a sink to wash hands and soap. Janitorial services are usually engaged once or twice a day to service the facility to do general maintenance. In medical field, once the bathroom is used, it is considered a contaminated space. As indicated by the survey, In general, consumers are more and more unhappy with the experiences in restrooms and it is becoming a cultural trend to ask for more active management of such spaces. Preferences appear to be that of cleaning after each use.

Systems do not currently exist which provide automatic cleaning of surfaces in stalls or bathrooms. Conventional cleaning approaches are only as effective as the person doing the cleaning. However, to ensure proper cleaning, the users can not be relied upon alone. Cleaning systems that clean without interaction of a user are unknown and not found.

Although strides have been made to provide better cleaning devices and techniques, shortcomings remain. A restroom sanitation and monitoring system is needed to permit a more thorough and complete cleaning of a restroom on demand. The system needs to be configured to allow both passive cleaning and active cleaning without the need for action on the part of the user.

### BRIEF SUMMARY OF THE INVENTION

An object of the present application is to provide a new, active restroom monitoring solution that is capable to monitor and sanitize surfaces and the air within a particular area, such as a restroom. The system is configured to provide real time sanitation, maintenance, and reporting to management of cleanliness levels within the space. They system is configured to be electronically controlled and provide tools and methods via a cleaning assembly to permit manual cleaning of surfaces by a user and to provide passive cleaning by the system of a plurality of surfaces and the air. Sanitation station can go further and bring additional conveniences to the user in the form of taste, smell, physical feel, visual aid, and touch—serving the basic human senses of the person.

The system is configured to utilize selectively times UV light to disinfect surfaces. One or more systems or sensors/modules are utilized to monitor cleanliness in the space and to report the status of the space.

It is an object of the present application to provide a cleaning assembly that includes a handle assembly configured to grasp a moistened tissue. A drum is included within a dispensing unit and configured to store the tissue. The dispensing unit selectively releases the tissue from the drum as an actuating assembly operates a lever between a first position and a second position. The drum is seated within the dispensing unit in communication with the actuating assembly. The cleaning assembly is designed to permit a user the ability to dispense a moistened tissue, grasp a moistened tissue, and clean with a moistened tissue without any contact with the moistened tissue or the selected cleaning surface. In other embodiments the dispensing of the tissue is automatic and controlled via a motor, thereby negating the need for the manual lever operation and assembly.

It is a further object that the cleaning assembly engage with the drum in a manner that retains the moisture in the tissues and prevents the drying out of the moistened tissue. The cleaning assembly may include one or more seals that are configured to automatically actuate to permit dispensing of tissue and then the subsequent sealing of the tissue cartridge to the outside air. In a manual configuration the cleaning assembly has a housing with a platform that seals around an opening of the drum. Also included is a moving platform that selectively seals beneath the housing platform. The moving platform disengages from the housing platform upon releasing of the moistened tissue.

Another object of the present application is to provide an actuating assembly that operates to disengage the moving platform from the housing platform prior to initiation of the rollers beginning the withdrawal of the moistened tissue. This prevents the moistened tissue from tearing. Movement of the lever arm in the actuating assembly first lowers the moving platform and then subsequently initiates the rollers as it moves from a first position to a second position.

The drum is configured to be interchangeable and optionally refillable with tissue.

The handle, when needed and used, includes a jaw that opens and closes via an input from a user. The jaw is designed to grasp the moistened tissue when dispensed from the dispensing unit. With the handle, the user is able to locate the tissue on a desired surface and scrub the surface by pressing a pad of the handle against the tissue on the surface. The hands of the user never have to contact the tissue at any time.

Another object of the present application is to provide a tissue dispensing unit for a wall that contains tissue. The unit includes a drum configured to store the tissue. The drum has an opening for passage of the tissue outside of the drum. The assembly further includes a housing configured to store the drum on a housing platform, the housing platform has a slot for the passage of the tissue external to the drum and includes a seal around the slot. The seal is configured to seal about the opening of the drum. Additionally, the unit includes an actuating assembly with a lever for initiating the dispensing of the tissue external to the housing.

The unit includes a moving platform configured to selectively seal against the housing platform and the slot. The lever initiates movement of the moving platform. Likewise, the actuating assembly includes a plurality of rollers configured to apply a force to the tissue so as to dispense it from the drum and externally to the housing. The plurality of rollers operating only after the moving platform disengages from the housing platform and slot.

The tissue dispensing unit may be mounted away from a wall such as on a stand to allow it freedom to sit in any location or be set on a counter or elevated surface.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a perspective view of a cleaning assembly according to an embodiment of the present application.

FIG. 2 is a side view of a handle assembly used in the cleaning assembly of FIG. 1.

FIG. 3 is an alternate side view of the handle assembly of FIG. 2, with a clamp in an open position.

FIG. 4 is a front perspective view of the handle assembly of FIG. 2.

FIG. 5 is a front perspective view of a dispensing unit used in the cleaning assembly of FIG. 1 with a front cover removed.

FIG. 6 is a side perspective view of the dispensing unit of FIG. 5 with a side cover removed.

FIG. 7 is a front lower perspective view of a drum in the cleaning assembly of FIG. 1.

FIG. 8 is a rear lower perspective view of the drum of FIG. 7.

FIG. 9 is a front perspective view of a housing of the dispensing unit of FIG. 5.

FIGS. 10 and 11 are enlarged front views of the housing of FIG. 9.

FIG. 12 is a front perspective view of the dispensing unit of FIG. 5, with a drum and side cover removed.

FIG. 13 is a top view of the dispensing unit of FIG. 12.

FIG. 14 is a lower perspective view of an actuating assembly in the dispensing unit of FIG. 12.

FIG. 15 is an upper perspective view of the actuating assembly of FIG. 14.

FIG. 16 is a top view of the actuating assembly of FIG. 14.

FIG. 17 is a lower view of the actuating assembly of FIG. 14.

FIG. 18 is a schematic of a sanitation and monitoring system using a variation of the cleaning assembly of FIG. 1, in accordance with the present application.

FIG. 19 is a front view of an embodiment of the sanitation and monitoring system of FIG. 18.

FIG. 20 is a schematic of an exemplary embodiment of a control unit in the sanitation and monitoring system of FIG. 18.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method in accordance with the present application overcomes one or more of the above-discussed problems commonly associated with elevated platforms discussed previously. In particular, the sanitation and monitoring system of the present application permits a user the ability to clean a potentially unclean surface without the need to physically contact any of the cleaning tissue, chemicals, or surface itself at any time. The user is separated from the potentially harmful surfaces and chemicals. The system is configured to include a cleaning assembly and utilize both active and passive methods of cleaning in an area. These and other unique features are discussed below and illustrated in the accompanying drawings.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The embodiments and method of the present application is illustrated in the associated drawings. The sanitation and monitoring system includes a cleaning assembly that may be either mechanically operated or electronically controlled. The manually operated cleaning assembly utilizes a manual lever to selectively dispense one or more moistened tissues. The electronically operated cleaning system automates dispensing of the moistened tissues and permits other passive cleaning functions. The cleaning assembly includes a handle assembly configured to grasp a moistened tissue. A drum or cartridge is included within a dispensing unit to store the tissue. The dispensing unit selectively releases the tissue from the drum as noted above. One or more modules and sensors are included to monitor the air cleanliness, report on cleanliness levels, and actively clean the air through UV lights and fans. Additional features and functions are illustrated and discussed below.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

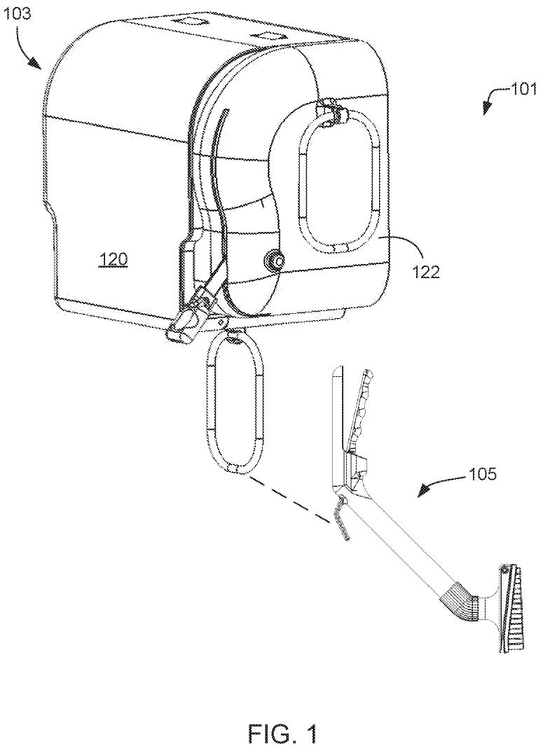

Referring now to FIG. 1 in the drawings, a perspective view of a manually operated cleaning assembly is illustrated. Cleaning assembly 101 includes a dispensing unit 103 and a handle assembly 105. Within the dispensing unit 103 is a plurality of tissues (not shown) that are selectively dispensed externally from the dispensing unit 103. Handle assembly 105 is configured to be obtained by a user and grasp the dispensed tissue and provide a means of exerting a force upon the tissue on a surface to be cleaned. The user is separated from the tissue, the cleaning surface, and any chemicals on the tissue at all times. The tissue may be discarded by operating the handle. As seen in FIG. 1, the handle assembly 105 includes a hook which permits it to rest on and/or hang from a portion of dispensing unit 103.

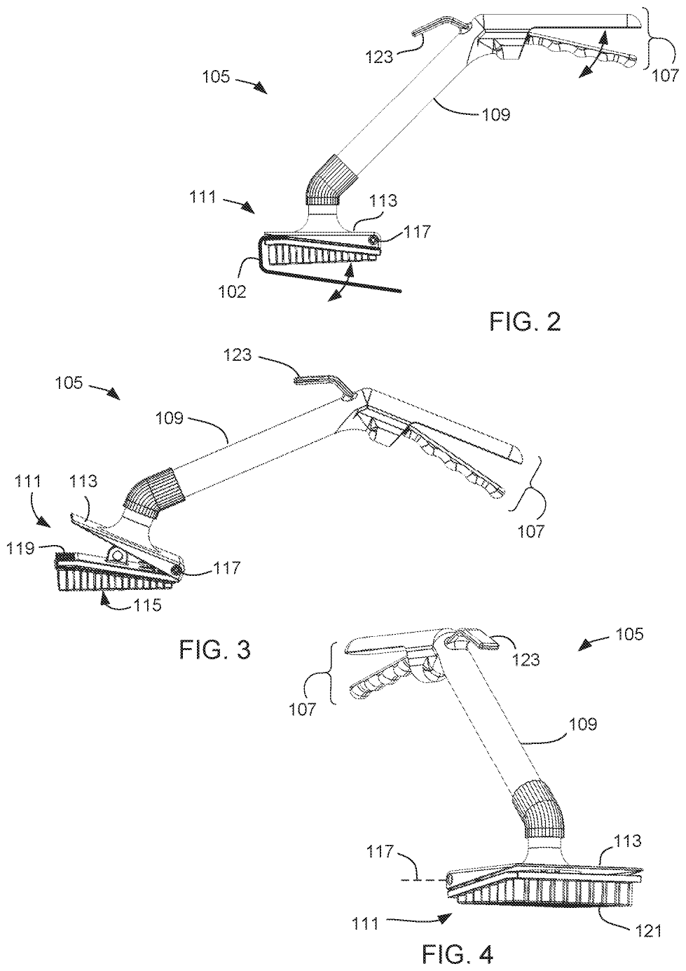

Referring now also to FIGS. 2-4 in the drawings, assorted views of handle assembly 105 are illustrated. Handle assembly 105 includes a handle 107, a shaft 109, and a clamp 111. Shaft 109 extends between handle 107 and clamp 111 and is useful to maintain separation of the user from the clamp 111. With handle 107, a user is able to grasp onto handle assembly 105 and operate clamp 111. Clamp 111 is configured to operate between an open and a closed position. In FIGS. 2 and 4, clamp 111 is closed. In FIG. 3, clamp 111 is shown in an open position.

At the base of shaft 109, clamp 111 includes a base 113 and a lower member 115. When handle 107 is actuated, lower member 115 pivots relative to base 113 along axis 117 so as to partially separate from one another. Along an upper surface of lower member 115 is a textured surface 119 to assist in grasping and holding the tissue 102. Once the tissue 102 is inserted into the open clamp 111, a user may release handle 107 and close clamp 111. The tissue is then laid onto a surface and a pad 121 along a lower surface of lower member 115 is pressed against the tissue 102 on the surface to be cleaned. As seen in the Figures, the hook 123 is located on shaft 109. It is understood that hook 123 may be located at any location on handle assembly 105 that adequately provides the capability of coupling with a portion of dispensing unit 103

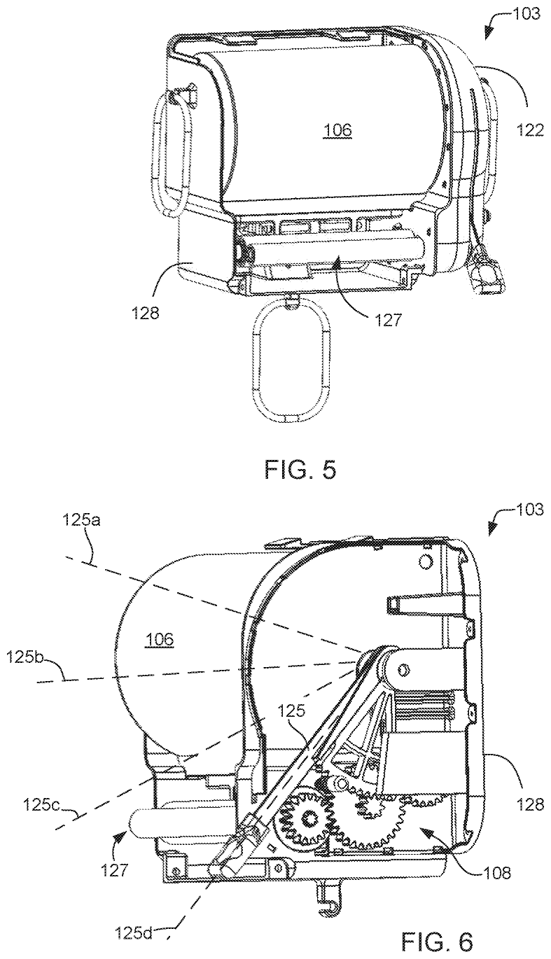

Referring now also to FIGS. 5 and 6 in the drawings, a front perspective view of dispensing unit 103 is shown with a front cover removed for clarity, along with a side perspective view with a side cover removed. Front cover 120 is removed in FIG. 5 to show the internal workings of unit 103. A drum 106 is seated internally therein. Within drum 106 is a plurality of tissues 102. The tissues are preferably moistened with a chemical of some type to assist in sanitizing and cleaning. However, the tissues may be dry.

As seen in FIG. 6, unit 103 includes an actuating assembly 108 configured to facilitate the dispensing of tissues from drum 106. A plurality of gears, shafts, linkages, and other components are included within actuating assembly 108, and are coupled to a housing 128. Each is used to help ensure proper operational timing of unit 103 such that the tissues are sealed/unsealed and/or dispensed in a proper sequence. In FIG. 6, side cover 122 is removed actuating assembly 108.

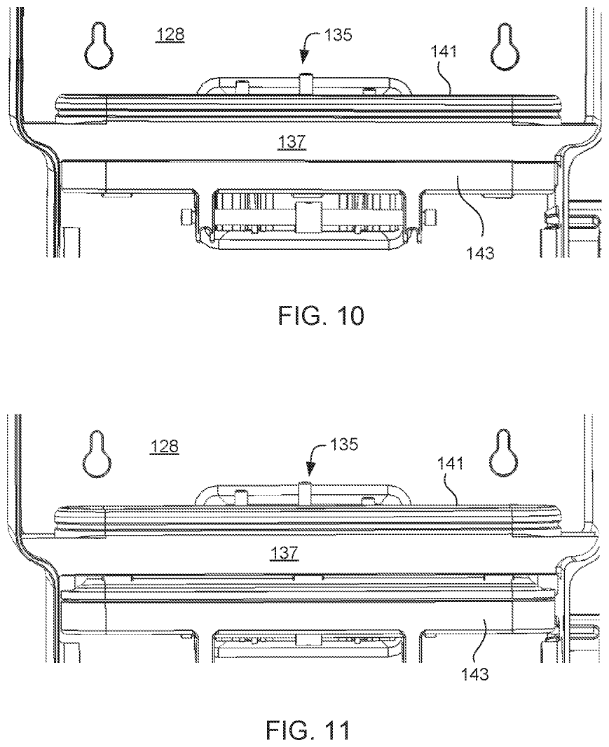

Assembly 108 includes a lever 125 operable between a plurality of positions. The positions are designated as positions 125a-125d. Position 125a is a neutral position used prior to initiating any dispensing action of tissues 102. From position 125a to position 125b, the drum and tissues are unsealed. From position 125b to position 125c, the rollers 127 of assembly 108 are activated and apply a force to the tissue 102 so as to dispense it from the drum 106 externally to unit 103. The rollers 127 rotate as lever 125 is in motion between positions 125b to 125c only. Further discussion related to the operation of assembly 108 with handle 124 will be provided later with FIGS. 10 and 11.

It is understood that although a lever is disclosed, it is only meant to be a single example of a manually operated unit 103. In other embodiments, unit 103 may utilize an electronic actuating assembly 108 having a motor and a motion sensor that can automatically sense motion and then dispense a predetermined amount of tissue. Additionally, a combination of the two types may be combined into a single embodiment with a button used to select between the two types. For purposes here, a manual operation will be discussed.

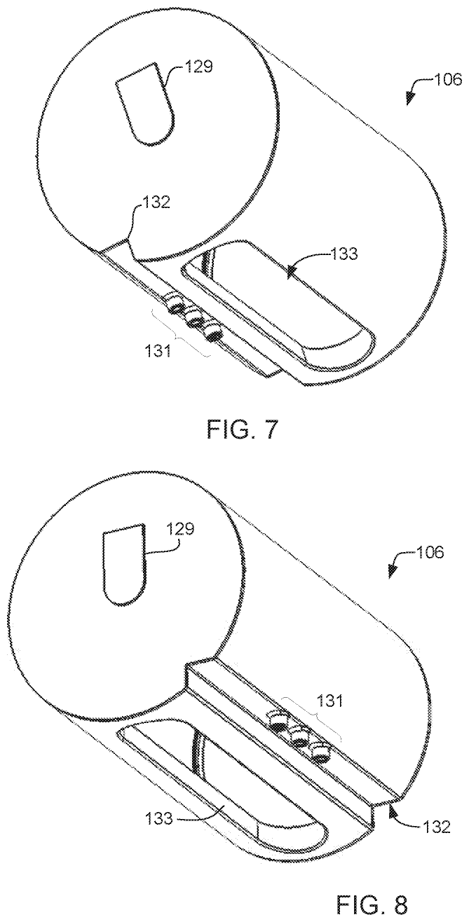

Referring now also to FIGS. 7 and 8 in the drawings, views of drum 106 are provided. Drum 106 is a cylindrical body having opposing ends that are capped off. Each end has a detent 129 extending outward to assist in proper alignment of drum 106 when seating within housing 128. A notched portion 132 exists on the lower area of drum 10. In the notched portion 132 is a keyed portion 131 having a series of protrusions extending from a surface thereof. The keyed portion 131 is unique to the type of drum 106 and is useful in restricting use of unit 103 to authorized or legitimate drums 106 from the original manufacturer. Drum 106 is interchangeable from housing 128. For example, when tissues 102 run out, drum 106 may be interchanged with a separate drum. It is understood that the keyed portion 131 may have any number of different protrusions. In one embodiment it is conceived that keyed portion 131 may consist of any type of device such as an electronic device that may emit a signal for authentication. Any manual, physical, and/or electronic keyed portion 131 is conceivable.

Additionally, drum 106 includes an opening 133. Opening 133 is aligned with detents 129 to permit function within housing 128. Tissues 102 pass from an internal location within drum 106 through opening 133.

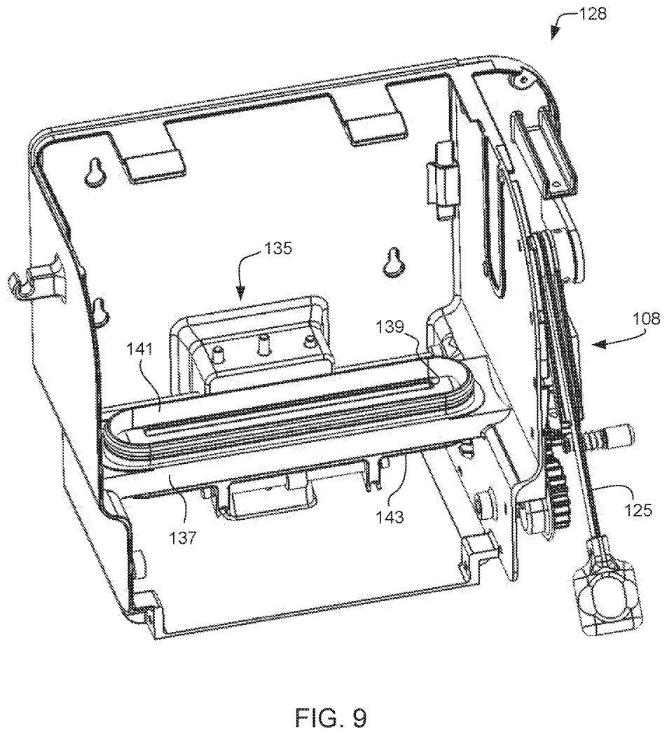

Referring now also to FIG. 9 in the drawings, a front perspective view of housing 128 is illustrated. In this Figure, drum 106 is removed for clarity. Of note is the mating portion 135 located internally. This is to match up with keyed portion 131. When the mating portion 135 and keyed portion 131 match, operation of actuating assembly 108 is permitted. In other words, operation of actuating assembly 108 is regulated by keyed portion 131 and mating portion 135 being a match or not.

Another feature of housing 128 is the use of a housing platform 137 extending from side to side of the internal cavity. Platform 137 includes a slot 139. Drum 106 is configured to align with platform 137 and slot 139 in particular. As tissues 102 leave drum 106, they are to pass through slot 139. Platform 137 also includes a seal 141 that rests on an upper surface of platform 137 and extends around slot 139. As drum 106 is located or seated within housing 128, opening 133 is seated within seal 141 above slot 139. Seal 141 is between platform 137 and drum 106. Seal 141 prevents moisture from leaving drum 106 and the drying out of tissues 102.

As noted previously, tissues 102 pass through slot 141, therefore a means of sealing slot 141 from the bottom of platform 137 is necessary. Actuating assembly 108 further includes a moving platform 143 in communication with lever 125 through one or more gears. As lever 125 actuates through positions, moving platform 143 is configured to move (i.e. pivot, rotate, translate, etc.). Moving platform 143 is configured to seat against a lower surface of platform 137 and seal around slot 139. This effectively seals out any air and moisture loss within drum 106.

Referring now also to FIGS. 10 and 11 in the drawings, enlarged front views of platform 137 and moving platform 143 are illustrated in different positions. In FIG. 10, moving platform 143 is located in a seated position against the lower surface of platform 137, thereby sealing out air from drum 106. In this position, lever 125 is in position 125a. In FIG. 11, moving platform 143 is shown in a lowered position. This occurs as lever 125 is moved to position 125b. Moving platform then stays in a lowered position until lever 125 is moved back to position 125a. Moving platform 143 remains lowered through positions 125b-125d. It is understood that the amount of movement of platform 143 can vary. Movement is necessary to release tissues 102 to allow rollers 127 to pull or dispense more tissue for a user. In position 125d, rollers 127 are not rotated and moving platform 143 is maintained in a lowered position. In this condition, drum 106 is more easily interchanged as slot 139 is open through platform 137. A new drum 106 may be inserted and tissue 102 passed through slot 139 prior to making its way through rollers 127. Once reloaded or in a ready to operate condition, lever 125 may then be rotated back up to position 125a ready for dispensing. Rollers 127 are not necessarily configured to rotate when lever 125 goes from positions 125d or 125c to 125a. rotation of rollers 127 would cause either more tissue 10 be dispensed or some tissue to be retracted. This is unnecessary although possible in selected configurations.

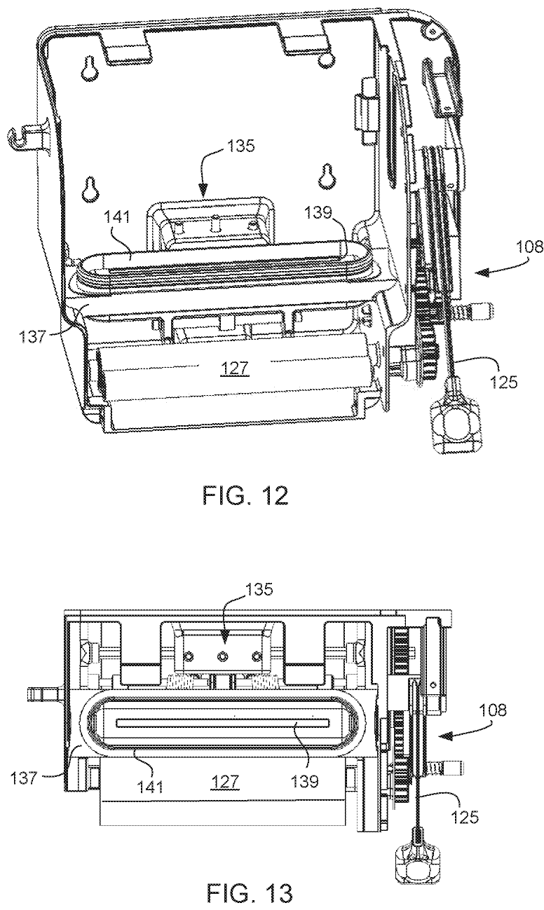

Referring now also to FIGS. 12 and 13 in the drawings, views of housing 128 with actuator assembly 108 and rollers 127 are provided.

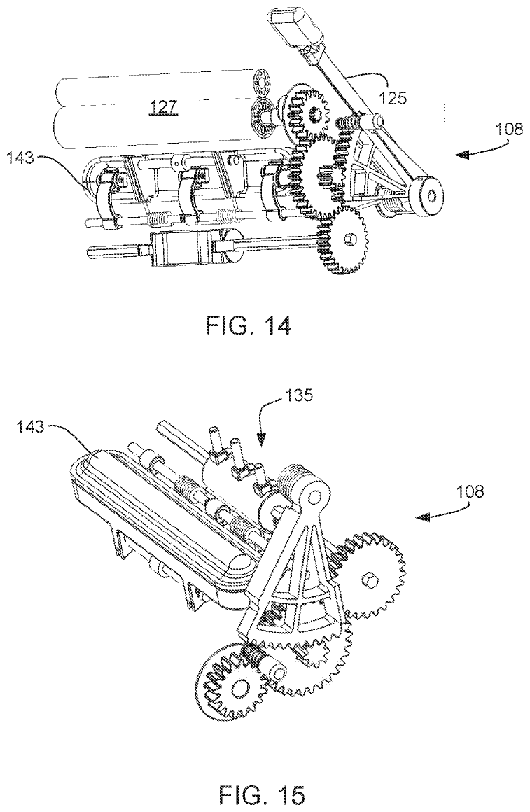

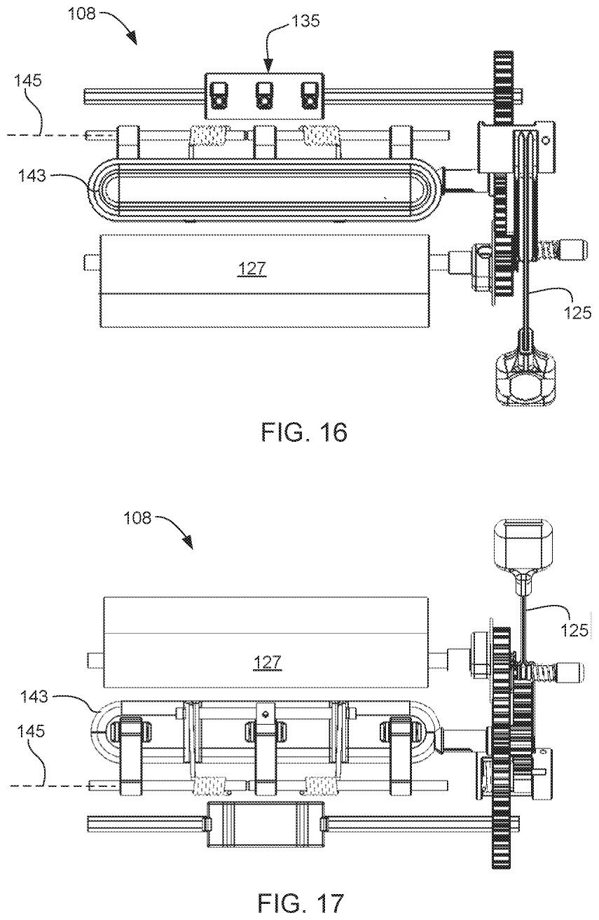

Referring now also to FIGS. 14-17 in the drawings, views of actuator assembly 108 are provided. As noted previously, actuator assembly 108 includes lever 125, a plurality of gears or mechanical actuation devices, moving platform 143, and rollers 127. As lever 125 changes positions, the gears sequentially operate the moving platform 143 followed by the rollers 127. Keyed portion 131 and mating portion 135 act as a security locking device to regulate movement of lever 125 and the operation of unit 103. As seen in FIG. 15 in particular, moving platform 143 is shaped so as to seat with and match the contour of platform 137. It is understood that other shapes are possible.

Lever 125 is ideally preloaded with a set resting position in the upper position of position 125a. This means transitioning into positions 125b and 125c will have some feedback moment on the user as lever 125 naturally wants to rest in position 125a. However, in position 125d, the feedback moment of lever 125 is done away to allow for interchanging. In the preferred embodiment, lever 125 naturally stays in position 125d when placed there and until a user changes its position. In other embodiment, lever 125 may be held there by a securing device as feedback moment may be felt in all positions. If left alone, lever 125 will transition to position 125a from positions 125b and 125c.

Moving platform 143 is configured to rotate about axis 145. Springs are coupled to the axis rod to ensure a preloaded pressure in the seated position. This pressure is overcome through movement of lever 125 to position 125b.

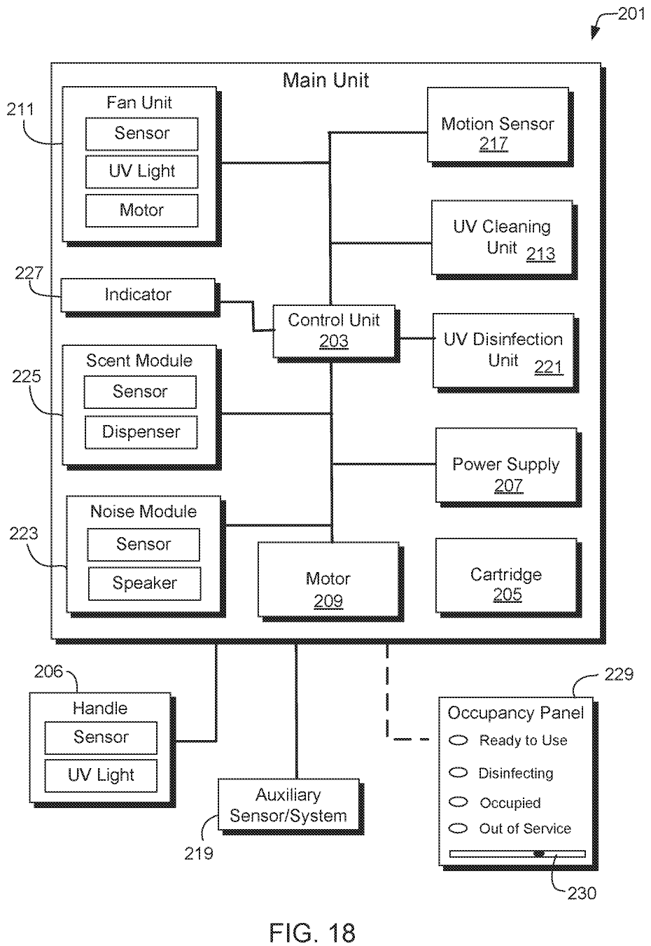

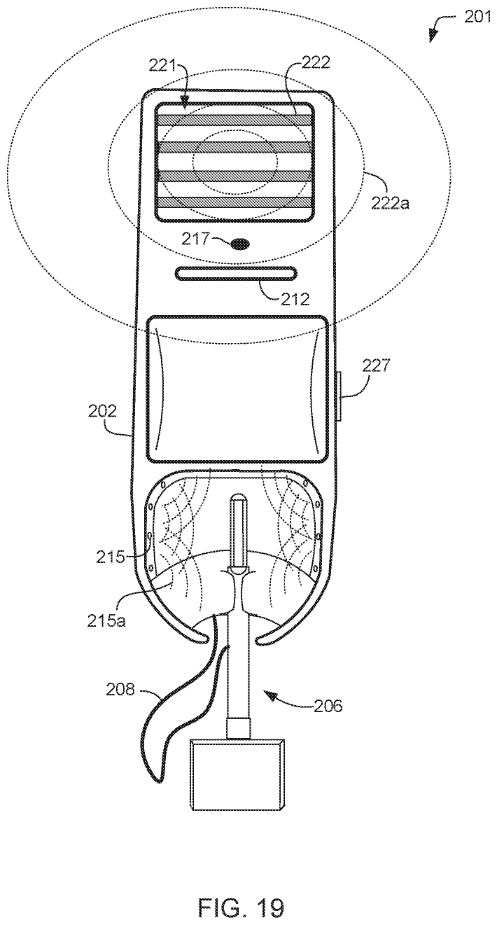

Referring now also to FIGS. 18 and 19 in the drawings, an electronically operated cleaning system, as noted by it being a sanitation and monitoring system 201, is illustrated. System 201 is similar in form and function to that of assembly 101. The purpose and function is the same in that a goal is to provide a user the ability to clean a surface without the need to contact either the wipe/tissue or the surface to be cleaned.

It is recognized that 70% of restroom users have unpleasant restroom experiences, while 23% of customers leave the establishment without completing business. Bathroom/toilet spaces are currently provided and built to meet only basic human needs, such as a toilet, toilet paper, water to wash hands/soap, along with a method to dry the hands. Janitorial services are usually engaged once or twice a day to service restrooms and to do general maintenance. System 201 is configured to provide communications links between maintenance personnel, customers/users, and other interested parties to relay information such as any of the following, status, occupancy, cleanliness level, needs for repair, and so forth. System 201 includes a control unit 203 configured to regulate the operations and communications of the parts therein.

It is understood that the functions of a drum/cartridge are included to hold moistened tissues, a handle is optionally included as an automated method of selectively dispensing the tissues is preferred with system 201. Whereas in assembly 101 a manual assembly was used, in system 201 an electronically controlled assembly is used. A motor is included to automatically dispense the tissues when needed. Other functions are permitted with the application of electrical power to further enhance the capabilities and functions of assembly 101 to make it into system 201. It is understood that any portion of the mechanically operated systems may be maintained in system 201 from assembly 101 and that system 201 may include any portion or all of the features herein described.

System 201 is intended to be a new, active restroom sanitation and monitoring solution to provide real time sanitation, maintenance, and reporting to management/users regarding at least one of status, occupancy, cleanliness level, repairs and so forth. Through system 201, a customer should feel that every time they entered the bathroom, they can expect that it was cleaned, sanitized, disinfected, and actively maintained. Management should be aware at all times of any issues with the bathroom in real-time and be able to either act on it or create set of rules for system 201 to execute it automatically. System 201 can go further and bring additional conveniences to the user in the form of taste, smell, physical feel, visual aid, and touch thereby serving the basic human senses of the person.

System 201 includes a central control unit 203 configured to regulate the performance of one or more of the systems, modules, sensors, units, or assemblies within system 201. A cartridge 205 similar in form and function to that of drum 106 and is ideally automated in its function through power supply 207 and motor 209. Supply 207 provides stored energy through one or more batteries or may be configured to include an outlet to provide power as needed through an external power source. Both A/C and D/C power are possible. One or more battery packs are possible. For instance individual components or parts of system 201 may include an individual battery pack or power supply, such as handle 206.

Motor 209 may be one or more motors that work collectively or independently to provide the functions of system 201. For example, motor 209 may operate fan unit 211 while an independent motor 209 may operate UV Cleaning unit 213. Motor 209 is referred to as a collective number of units, namely a single motor or multiple motors. Motor 209 is configured to activate the dispensing of tissue from cartridge 205.

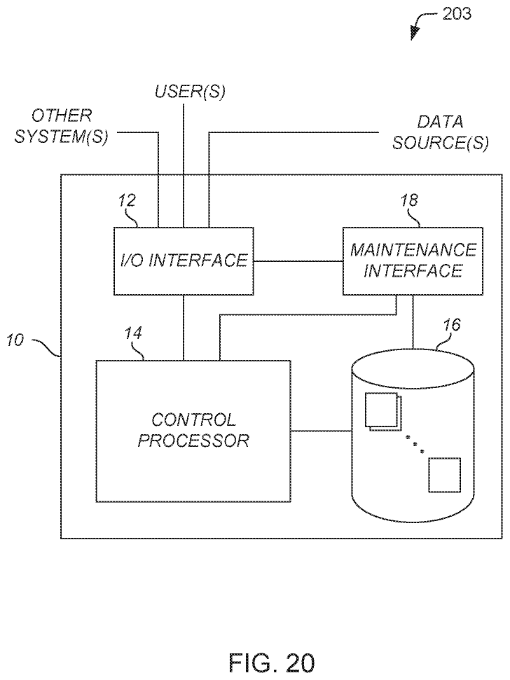

Referring now also to FIG. 20 in the drawings, an exemplary schematic of control unit 203, as a generalized computer system, is illustrated. System 201 includes program software executed on one or more computerized devices. FIG. 12 illustrates an exemplary computerized device configured to execute and access the programs and function associated with system 201. It is understood that any other module, system, unit, or sensor illustrated within FIG. 18 may also include one or more components and functions represented in FIG. 20.

The system 10 includes an input/output (I/O) interface 12, a control processor 14, a database 16, and a maintenance interface 18. Alternative embodiments can combine or distribute the input/output (I/O) interface 12, control processor 14, database 16, and maintenance interface 18 as desired. Embodiments of the system 10 can include one or more computers that include one or more processors and memories configured for performing tasks described herein below. This can include, for example, a computer having a central processing unit (CPU) and non-volatile memory that stores software instructions for instructing the CPU to perform at least some of the tasks described herein. This can also include, for example, two or more computers that are in communication via a computer network, where one or more of the computers includes a CPU and non-volatile memory, and one or more of the computer's non-volatile memory stores software instructions for instructing any of the CPU(s) to perform any of the tasks described herein. Thus, while the exemplary embodiment is described in terms of a discrete machine, it should be appreciated that this description is non-limiting, and that the present description applies equally to numerous other arrangements involving one or more machines performing tasks distributed in any way among the one or more machines. It should also be appreciated that such machines need not be dedicated to performing tasks described herein, but instead can be multi-purpose machines, for example computer workstations, that are suitable for also performing other tasks. Furthermore the computers may use transitory and non-transitory forms of computer-readable media. Non-transitory computer-readable media is to be interpreted to comprise all computer-readable media, with the sole exception of being a transitory, propagating signal.

The I/O interface 12 provides a communication link between external users, systems, and data sources and components of the system 10. The I/O interface 12 is in communication with the control processor 14 and database 16 and is configured to provide an interactive link between system 201 and any remote third party. The I/O interface 12 can be configured for allowing one or more users to input information to the system 10 via any known input device. Examples can include a keyboard, mouse, touch screen, microphone, and/or any other desired input device. The I/O interface 12 provides a display portal defining a plurality of visually perceptible elements corresponding to the prediction data. The I/O interface 12 can be configured for allowing one or more users to receive information output from the system 10 via any known output device. Examples can include a display monitor, a printer, a speaker, and/or any other desired output device. The I/O interface 12 can be configured for allowing other systems to communicate with the system 10. For example, the I/O interface 12 can allow one or more remote computer(s) to access information, input information, and/or remotely instruct the system 10 to perform one or more of the tasks described herein. The I/O interface 12 can be configured for allowing communication with one or more remote data sources. For example, the I/O interface 12 can allow one or more remote data source(s) to access information, input information, and/or remotely instruct the system 10 to perform one or more of the tasks described herein.

The database 16 provides persistent data storage (computer readable storage media, i.e. hardware) for system 10. Database 16 is in communication with control processor 14 and I/O interface 12. While the term “database” is primarily used, a memory or other suitable data storage arrangement may provide the functionality of the database 16. In alternative embodiments, the database 16 can be integral to or separate from the system 10 and can operate on one or more computers. The database 16 preferably provides non-volatile data storage for any information suitable to support the operation of the system 10, including various types of data necessary to perform the functions and feature discussed below.

The maintenance interface 18 is configured to allow users to maintain desired operation of the system 10. In some embodiments, the maintenance interface 18 can be configured to allow for reviewing and/or revising the data stored in the database 16 and/or performing any suitable administrative tasks commonly associated with database management. This can include, for example, updating database management software, revising security settings, and/or performing data backup operations. In some embodiments, the maintenance interface 18 can be configured to allow for maintenance of the control processor 14 and/or the I/O interface 12. This can include, for example, software updates and/or administrative tasks such as security management and/or adjustment of certain tolerance settings.

The control processor 14 can be configured to perform a process or a plurality of processes such as the processes described below in connection with the associated Figures. Additionally, control processor 14 includes software programmed to compile properly communicate with the interested parties. Processor 14 includes a non-transitory computer-readable medium with instructions stored thereon to execute predetermined steps.

Referring back to FIGS. 18 and 19 in the drawings. Control unit 203 is configured to be at the heart of system 201 and is configured to regulate the performance of the entire system. System 201 as a whole is configured to monitor and automatically sanitize surfaces and/or a volume of air within a defined area. Communications may be transmitted out from and received by unit 203 via its interface. Such communications may be provided to third parties such as a potential user of a restroom for example or even that of an owner or maintenance worker for the facility. Unit 203 is configured to convey sufficient data to third parties about the conditions within the restroom or facility space.

System 201 includes a main body 202 to centralize and house the various components within the system. System 201 may be located in any general space within the defined area but it is preferred that it may be located in a manner that permits it to have a line of sight to heavily contaminated areas or places. Locations may include the toilet, the sink, a urinal, selected walls, or even a door for example. Control unit 203 is configured to communicate and regulate one of a number of modules, sensors, units, or systems to fully utilize the purpose of system 201.

As stated previously with respect to system 101, a handle 206 is included incorporating the functions and features of handle 105 seen via FIGS. 1-4 of the drawings. Handle 206 includes a tether 208 to secure handle 206 to a portion of body 202. Tether 208 may be retractable in some embodiments. It also may serve as a path for routing power to handle 206. Body 202 includes a harness or seat area that is configured to accept handle 206 between uses. It is understood that there are many different styles and methods of securing handle 206 via a harness or seat and the manner depicted is not meant to be limiting. It is desired that the grip area of handle 206 be oriented to be adjacent to or within the confines of body 202.

System 201 is configured to clean and/or sterilize one or more surfaces. This can be done manually through the use of handle 206 and a tissue in cartridge 205. Additionally system 201 incorporates the ability to automatically clean and/or sterilize through ultraviolet sources, which can emit ultraviolet radiation directed onto the surface(s). This does not need a user's manual interference. As sterilization or cleaning is possible through ultraviolet sources, body 202 is configured to include a UV cleaning unit 213. It is desired that surfaces around the defined area are cleaned for a new user. This extends also to the portions of system 201 that are interacted with by a user. Handle 206 is handled by a user and therefore should ideally also be cleaned. Cleaning unit 213 includes a plurality of emitting ultraviolet emitters 215 configured to broadcast UV radiation/light 215a onto surfaces of handle 206. Any and all surfaces of handle 206 may be cleaned via emitters 215. Other parts of body 202 may also be cleaned through emitters 215.

Control unit 203 is configured to regulate the operation or timing of use for emitters 215. It is understood that a user may be permitted to activate emitters 215 through a manually operated interface. In other preferred methods, unit 203 automatically detects the removal and replacement of handle 206 and operates emitters 215 via a time delayed function. There may exist some concerns over safety of users around UV lights especially where such operation is unsupervised. Such may be the case in restrooms with a number of stalls wherein each stall may include system 201. People in neighboring stalls may be tempted to look over or under a stall wall to see what is going on. A protector or barrier may be included coupled to the stall walls to prevent curious onlooking. The barrier may be tinted to avoid damage from the lights but otherwise permit viewing therethrough. Any number of types of devices may be used to obstruct viewing. Coupling to the stall walls may be done through customary methods and permit removability.

In a further embodiment, system 201 may include motion sensor 217 configured to detect motion in the defined area. Unit 203 may be configured to delay operation of emitters 215 until after the user has left the defined area. System 201 is configured to monitor the location of the user in the defined area and selectively alter or adjust the timing of emitters 215. Sensor 217 is just one manner in which system 201 may accomplish this.

System 201 may further include an auxiliary sensor/system suite 219. Suite 219 may be located either on or off of body 202. This system can include one or more different sensor used with control unit 203 to determine the location of a user in the defined area. Exemplary sensors are thermal sensors, infrared sensors, movement sensors, sound sensors, and so forth. These may be integrated into body 202 or be located in a remote distal location away from body 202. It is known that sensor 217 may communicate with one or more sensors in suite 219 along with unit 203 to determine the location of the user in the defined are.

System 201 also includes a UV disinfection unit 221. Unit 221 is configured to includes a plurality of emitting ultraviolet emitters 222 configured to broadcast UV radiation/light 222a onto surfaces external to that of body 202. Such surfaces may be that of the toilet, a sink, the floor, a wall, a door, or even handle 206 for example. The operation and timing of emitters 222 are done similarly to that of emitters 215 within unit 213 discussed above. Sensor 217 along with optional suite 219 may be used in communication with that of control unit 203 to regulate the timing and duration of operation of emitters 222.

It should be understood that the timing of emitters 215 and 222 are such that they operate when a user is not in the defined area. The defined area may be a bathroom stall, a hallway, any room needed to remain sanitary and so forth. System 201 is aptly configured to monitor the space within the defined area and automatically regulate performance, either on or off, of any system within itself. Ideally, operation of emitters 222 will occur after the user or any and all users have left the defined area (i.e. left the stall).

Discussion has been had regarding sensor 217 and it is understood that sensor 217 can activate any number of functions on system 201 as it is configured to analyze external user activity. System 201 is configured to clean surfaces in the space of the defined area. One manner in which it does this is through UV radiation/light which is not always suited for human exposure. From input of sensor 217, unit 203 is configured to determine the presence of a user in the defined area (i.e. present or absent), in which case selected features may be functional.

Fan unit 211 is configured to circulate air through body 202 all while subjecting it to UV radiation/light so as to sanitize or clean the air. Air may be pulled in and/or released through a port 212 in body 202. Unit 211 may include a separate motor to that of motor 209 and includes one or more emitters to disseminate the UV light. Air is sucked through port 212 and is fed into a UV panel with the one or more UV lights to destroy airborne pathogens.

UV capabilities has been shown to exist with fan unit 211 to clean the air, disinfection unit 221 to clean remote or distal surfaces in the defined area, and cleaning unit 213 to disinfect surfaces on handle 206. It is noted that handle 206 may further include a sensor that detects the cleanliness level on its own surfaces and a UV light emitter that can automatically clean its own surfaces. In this embodiment, handle 206 is not dependent upon unit 213 only. Taking the UV capabilities to the next level would be the feature that handle 206 allows its own UV capabilities to be broadcast externally to other surfaces. For example, handle 206 may emit UV light to surfaces adjacent handle 206 for the purpose to clean and sanitize. This would permit hard to reach areas a way to be disinfected. Handle 206 thereby creates direct UV application to the surface by placing itself in proximity with such areas.

It is recognized that any of the UV features and devices in system 201 may be interchangeable and adjustable to allow for orientation changes and size changes to permit proper coverage in the defined area. The UV light can have any desired wavelength. For example, the wavelength used in system 201 may be anywhere between 220 nm and 400 nm.

Other optional feature of system 201 is the inclusion of a noise module 223 and a scent module 225. Noise module 223 is configured to broadcast an overlapping or suppressing noise to cancel out other noises in the defined area. Often a user may desire privacy from noises in the bathroom and not want to broadcast those noises themselves. Unit 203 can monitor noise in the defined area and issue any other noise through a speaker. The noise module 223 may include a sensor to detect noise, volume, and so forth. The noise generated may be random, may serve as white noise, may be noise cancelling, or merely be a noise to mask other noises generated by the user. These may be activated by a user manually through an interface on body 202 or through an electronic device. Alternatively, system 201 may link operation of noise module 223 with motion sensor 217 where it automatically is activated, or even through voice activation.

Scent module 225 is configured to operate similarly to that of module 223 in how control unit 203 regulates operation. It may be activated by a user manually through an interface on body 202 or through an electronic device. Alternatively, system 201 may link operation of scent module 225 with motion sensor 217 where it automatically is activated, or even through voice activation. Module 225 is configured to selectively release an odor or scent into the air of the defined area to mask or overpower undesired smells with a more pleasant one. This helps to avoid embarrassment of the user on the toilet. This odor neutralizing effect works through sensor to monitor and detect smells along with a dispenser to release those smells into the air.

System 201 may further include an indicator 227 visually viewable from outside body 202. Indicator 227 is configured to convey through visual representation a status of at least one of the area and its cleanliness and the status of system 201. One or more lights may be used. Alternatively, switches, knobs, or mechanical items may be incorporated to convey a visual message of status. A plurality of indicators may be used. Indicator 227 may provide the user with information related to any of the following exemplary items: service needs of system 201, disinfection in progress, needs further disinfection, ready for use, and so forth.

An occupancy panel 229 may also be included. Panel 229 differs slightly from that of indicator 227 in that it is ideally detached from body 202. Panel 229 may be located distal from body 202 and located in remote places, such as places of entrance into the defined area. For example, panel 229 may be located on the door into a bathroom stall. Panel 229 may be wired or have wireless capabilities to communicate with control unit 203. Panel 229 may include simple LED indicators or a digital panel with live text or images. This would allow any person entering the bathroom space to see information such as “Ready for Use”, “Disinfecting in Progress”, “Occupied”, “Out of Service”, for example. Such features are important so that any person going to use the toilet can see its current status. A progress bar 230 may be also included to show an instantaneous status or progress through a task performed by the system 201, such as cleaning.

Panel 229 may be located in a more open entrance to the restroom where it may serve to tell people waiting in a line for access, which stall is available. A waiting user may be able to activate a button on panel 229 or other location that will send a signal to the person occupying a toilet that there is a person waiting in line. The user inside the door will get either visual or voice notification. In some instances, instead of just using small indicators on a door, system 201 may project colored lights to the ceiling/and/or floor so that users standing in a line can see the status of the stall. This helps user determine from a distance status of the stall.

Although various functions and systems have been described herein with respect to system 201, it is important to note that there are other nuances and features in how the system as a whole operates together to accomplish the task of keeping the defined area clean and sanitized. One such feature is the option for control unit 203 to regulate the operation of any UV light emitting devices to be set in accordance with a timer. The timer may be set to allow sufficient exposure to ensure a clean area. This time may be adjusted automatically. One such reason to provide the ability to adjust the time is to adapt to different types of bulbs that may be used to emit the UV light. LED bulbs usually require significantly longer times to disinfect while florescent bulbs require shorter periods of time. The user may manually make the time adjustment or unit 203 may automatically make the change upon notification of the type of bulb or through detection of the bulb type.

Auxiliary system 219 may also be a robotic arm wherein it articulates to manually clean a surface such as a toilet. The function of the robotic arm is similar to wand 206 but it different in that it is operated through control unit 203 to selectively come into contact with the remote surface. System 219 may also be auxiliary UV light panels that operate with any other UV emitting system of system 201. These may be remotely located in the defined area to aide in reaching all surfaces of concern. The remote panels would be in communication with the respective units in body 202 and/or through unit 203.

System 201 may be configured to use sensor 217 and/or system 219 to assess or measure the space of the defined area. This is useful for operation of the UV functions. It may use optical and other sensors to calculate room size and dimensions to apply appropriate levels of UV light for disinfection.

System 201 may also include an alarm system through unit 203 wherein a noise may be emitted or a communication sent via an interface to signal that tampering or vandalism has occurred. This may apply to the system 201 itself or to the defined area through recognition via the one or more sensors. Various sensors can be in place allowing to track items being disconnected, being hit and generate movement, or if an item is taken away from the station at a certain distance (proximity sensors). In Some cases handle 206 may use this application as it's not always convenient to have security cord attached to it.

System 219 may also include a door sensor to allow unit 203 to communicate if a door to the defined area is closed or open. The sensor may be located in any portion of the door or be aligned with the door and rely on visual sight of the door to make determination of its open and closed status. Such sensors can be installed in door locks, hinges, or variety of other places to determine that the door is open or closed and even selectively regulate access through the door. Remote Door open/Close. It is conceived that when a problem arises with the system 201 or with the defined area, a maintenance person doesn't have time to run and mark the restroom unavailable. In this scenario, then user or unit 203 communicates an issue (i.e. bathroom unsanitary for use, or if some essential items are out of stock like “toilet paper”), the remote person like the maintenance person may close and/or lock the door remotely.

System 219 may also be a light sensor configured to scan a surface to determine if it is sanitary or not. If dirt is detected, system 201 may selectively activate one or more cleaning functions as described herein to rectify the problem. In some embodiments, system 201 may communicate with a smart toilet to allow unit 203 to regulate flushing of the toilet.

Sensors of different types can be associated with auxiliary sensor system 219. For example, cigarette, smoke, and drug detection sensors are possible. In schools, airports, or other public bathrooms sometimes it's important to track usage of smoke or drug related activity. Sensors can be installed to report to an administrator through unit 203. Another sensor is a sensor configured to scan floors for foreign trash, dirt, debris, and report issue to administrator. Likewise a sound sensor is conceivable to detect sneezing or coughing of the user in the defined area. As viruses and infections are often spread through the air in this manner, system 201 may selectively take action tailored to the type of sound it hears to clean the defined area. Other items such as robotic cleaning systems to clean the surfaces or robotic vacuum cleaners are feasible. Furthermore, sensors to track temperature in the defined area that communicate with air conditioning and heating systems are possible. Likewise, a heat pump may be included to provide heat to the toilet seat to increase comfort of the user. Auxiliary sensor/system 219 can be any device that works in communication with system 201 to provide cleaning or assist is assessing cleanliness and comfort of the user in the defined area (i.e. video screens to relay messages). It is conceivable to integrate system 201 into a toilet to save space.

It is noted that all features, individually or collectively, within system 201 may be controlled by voice dictation. Commands may be provided by a user in the defined area to either convey a message to a third party or activate a specific operation. Commands or voice dictations may be as follows: more paper, Disinfect, Stop Disinfection, Apply Deodorant, etc. In selected embodiments, system 201 may be configured to dispense candy or mints to help user freshen up.

Control unit 203 is an electronic system that regulates the functions of system 201. It may include automated software responses based on set logic. Responses may be automated and predetermined to set situations or inputs. In other examples, artificial intelligence may be applied to preemptively anticipate situation prior to occurring and allow system 201 as a whole to adapt to conditions and learn.

As noted previously, unit 203 includes an interface that allows it to communicate with other devices in system 201 and with third parties either through wired or wireless communications. Unit 203 may convey countless types of information such as the current status on any and all features and functions of system 201 and on the defined area. This information can be sent to future end users via an app in some situations to help future users make a decision to visit or not to visit a particular stall. Voice capturing through unit 203 is possible through one of its internal input/output interfaces to allow a user the speak to it and then unit 203 record the information received for transmission.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A cleaning system, comprising:
a control unit within a body;
a cartridge configured to store a tissue within the body, the cartridge being located within a body;
a motor in communication with the cartridge and configured to selectively dispense the tissue;
a handle configured to manually grasp the tissue for cleaning; and
a UV light emitting system coupled to the body for selectively broadcasting UV light to clean a surface adjacent the body.

2. The system of claim 1, wherein the UV light emitting system is a UV cleaning unit configured to emit UV light onto a surface of the handle.

3. The system of claim 1, wherein the UV light emitting system is a UV disinfection unit configured to emit UV light external to the body on a remote surface.

4. The system of claim 1, wherein the control unit regulates the timing and duration of the UV light emitting system.

5. The system of claim 1, wherein the control unit is configured to capture and relay communications from the user in a defined area and transmit them to a third party.

6. The system of claim 1, wherein the control unit is configured to monitor and detect at least one of a sneeze and a cough by a user in a defined area.

7. The system of claim 1, further comprising:
a scent module to monitor odors in the air and selectively release a scent.

8. The system of claim 1, further comprising:
a noise module configured to monitor noise in a defined area and selectively produce a sound in response to the noise detected.

9. The system of claim 1, further comprising:
a motion sensor configured to scan a defined area and detect the presence of a user.

10. The system of claim 9, wherein the control unit selectively activates and deactivates the UV light emitting system based upon the detected presence of the user.

11. The system of claim 1, wherein the handle includes a UV light, the UV light emits a light onto a surface of the handle for cleaning

12. The system of claim 1, wherein the handle includes a UV light, the UV light emits a light onto a remote surface for cleaning, the UV light being emitted manually.

13. The system of claim 1, wherein the handle includes a retractable tether coupled to the body.

14. The system of claim 1, further comprising:
a panel located remote from the body, the panel being configured to monitor the status of a defined area and communicate that status to a user or third party.

15. The system of claim 1, further comprising:
a fan unit configured to circulate air within a defined area, the fan unit including a UV light emitting bulb to clean the air.

16. The system of claim 1, further comprising:
an auxiliary sensor system in communication with the control unit, the auxiliary sensor system includes a door sensor to track the status of a door into a defined area as being open or closed, the door sensor being configured to operate a door lock to selectively permit access.

17. The system of claim 1, further comprising:
an auxiliary sensor system in communication with the control unit, the auxiliary sensor system includes a sensor to scan surfaces within a defined area for cleanliness, the control unit selectively initiating a cleaning function in response.

18. The system of claim 1, further comprising:
an auxiliary sensor system in communication with the control unit, the auxiliary sensor system includes a robotic arm operated through the control unit to selectively articulate into contact with a remote surface.

19. The system of claim 1, further comprising:
an auxiliary sensor system in communication with the control unit, the auxiliary sensor system includes UV light panels selectively located throughout the defined area, the UV light panels configured to emit a UV light.

20. The system of claim 1, further comprising:
an auxiliary sensor system in communication with the control unit, the auxiliary sensor system includes an infrared sensor, the control unit processing inputs from the infrared sensor to determine the presence of a user in the defined area.
